(19) United States
(12) Patent Application Publication
(10) Pub. No.: US 2022/0362080 A1
(43) Pub. Date: Nov. 17, 2022

(54) Electrically Active Hospital Bed Caddy with Adjustable Mobile Device Mount and Cable Harness

(71) Applicant: Arbor Devices LLC, Chicago, IL (US)

(72) Inventors: Michael Shannon McCorquodale, Chicago, IL (US); Sam Backes, Newark, CA (US)

(21) Appl. No.: 17/739,710

(22) Filed: May 9, 2022

Related U.S. Application Data
(60) Provisional application No. 63/187,642, filed on May 12, 2021.
(60) Provisional application No. 63/187,687, filed on May 12, 2021.

(51) Int. Cl. A61G 7/05; A61B 50/22

(52) U.S. Cl. CPC A61G 7/0507; A61B 50/22

(57) ABSTRACT

A representative embodiment of a hospital bed caddy is disclosed, comprising: a housing having a base panel with a first opening and one or more side panels coupled to the base panel to form a plurality of compartments, including first and second compartments; a bed rail attachment; a plurality of electrical interfaces arranged in the second compartment; a plurality of electrical cables arranged in the first opening, with each electrical cable coupled within the second compartment to a corresponding electrical interface; and a cable harness or sheath coupled around the plurality of electrical cables. A cover panel may be included, with the cover panel separating the second compartment from the first compartment, and with the electrical interfaces being user-accessible from within the first compartment. Various embodiments may also include one or more slots in the various panels and a moveable extension arm with a mobile device mount.

### CROSS-REFERENCE TO RELATED APPLICATIONS

This application is a nonprovisional of and claims the benefit of and priority to U.S. Provisional Patent Application No. 63/187,642, filed May 12, 2021, inventors Michael Shannon McCorquodale et al., titled “Electrically Active Hospital Bed Caddy with Adjustable Mobile Device Mount and Wire Harness”, and is a nonprovisional of and claims the benefit of and priority to U.S. Provisional Patent Application No. 63/187,687, filed May 12, 2021, inventors Michael Shannon McCorquodale et al., titled “Electrically Active Hospital Bed Caddy with Adjustable Mobile Device Mount and Wire Harness”, all of which are hereby incorporated herein by reference in their entireties with the same full force and effect as if set forth in their entireties herein.

### FIELD OF THE INVENTION

The present invention, in general, relates to bed storage caddies and, more specifically, relates to a hospital bed caddy having electrically active charging and communication interfaces with a mobile device mount and a wire harness.

### BACKGROUND OF THE INVENTION

One of the walls in a typical hospital room, which may be referred to as a “head” wall, includes a wide array of devices for providing medical treatments and services, including oxygen or other air lines for providing supplemental oxygen to a patient, a vacuum device and container for providing suction, electrical outlets for providing alternating current (AC) power (such as for providing power to an intravenous (IV) pump and various patient biometric monitors), a telephone (landline) communication interface (such as a registered jack 11 (RJ-11 jack)), and a communication and control interface or connector for plugging in an electrical cable or cord of a patient remote control device, such as a Deutsches Institut für Normung (DIN) interface. Such a patient remote control device typically provides a variety of functions, including providing an alert to the nurses' station (such as to summon a nurse or other care provider), providing a microphone and speaker for communication with the nurse or other care provider, providing controls for a television (such as power on/off, volume and channel selection), and may also provide control over positioning of portions of the bed (such as raising and lowering the head or the foot of the bed).

The hospital bed typically has multiple rails on each side of the bed which may be raised and lowered, as needed. The hospital bed is typically positioned generally perpendicular to the head wall with the head of the bed arranged adjacent to the head wall. In many circumstances, there is no place to store the patient remote control device within reach of the patient, so the patient remote control device is placed on the patient bed itself. A care giver may also wrap the cable of the patient remote control device around and through the railings of the hospital bed, so that if the patient remote control device falls from the bed, the patient can still grasp the cable and retrieve the patient remote control device.

One of the difficulties experienced by patients and medical staff is the lack of organization and storage for many of the devices in a hospital room. For example, a patient typically has one or more mobile devices, such as a mobile cellular telephone, a mobile tablet, a laptop, or a gaming device, any and all of which may need to be charged periodically. There is often no place to store the mobile device while it is being charged from the head wall, as the charging cords often do not extend close enough to the patient bed for the device to be within easy reach while plugged in to the head wall. In addition, the patient tray table is typically in use and filled with other items, such as a food tray, water, cups, drinking glasses, a box of tissues, books, magazines, and so on. Moreover, all of these multiple electrical cords from the head wall may become entangled, not only with each other but also with the various medical tubes, lines and catheters connected to the patient. All of these multiple electrical cords connected to the head wall further make it more difficult to move equipment over and around these cords, such as to move the IV pump (on a moveable, wheeled stand) as the patient is moving around the hospital room.

Accordingly, a need remains for a convenient hospital bedside solution to improve the patient experience by facilitating organization and management of hospital and personal devices and cables in a hospital room. Such a hospital bedside solution should provide for charging of mobile devices and for storage of such mobile devices, the patient remote control device, and other personal items, all within easy reach of the patient. Such a hospital bedside solution should also be compatible with existing hospital infrastructures and should avoid any potential interference with hospital devices and treatments.

### SUMMARY OF THE INVENTION

The exemplary or representative embodiments of the present invention provide numerous advantages. Various representative embodiments provide a hospital bed caddy having electrically active charging and communication interfaces with a mobile device mount and a cable harness. The representative embodiments improve the patient experience by facilitating organization and management of hospital and personal devices and cables in a hospital room. The representative embodiments provide for charging of mobile devices and for storage of such mobile devices, the patient remote control device, and other personal items, all within easy reach of the patient. The representative embodiments are also compatible with existing hospital infrastructures and avoid any potential interference with hospital devices and treatments.

In a representative embodiment, a hospital bed caddy is provided, comprising: a housing comprising: a base panel having a first opening; and one or more side panels coupled to the base panel to form a plurality of compartments, the plurality of compartments comprising a first compartment and a second compartment; a bed rail attachment coupled to or integrally formed with the housing; a plurality of electrical interfaces arranged in the second compartment; a plurality of electrical cables arranged in the first opening, each electrical cable of the plurality of electrical cables coupled within the second compartment to a corresponding electrical interface of the plurality of electrical interfaces; and a cable harness or sheath coupled around the plurality of electrical cables.

A representative embodiment may also include a cover panel having one or more second openings, with the cover panel coupled over the plurality of electrical interfaces to separate the second compartment from the first compartment. As such, the plurality of electrical interfaces are user-accessible from within the first compartment.

Representative embodiments of the hospital bed caddy may also include additional compartments; for example, the base panel and the one or more side panels may further form a third compartment, of the plurality of compartments, the third compartment arranged adjacent to the first compartment. In a representative embodiment, a portion of the base panel and at least one front side panel of the one or more side panels of the third compartment further comprise a slot or slot opening, such as to accommodate an electrical cable from a patient remote control device.

In a representative embodiment, the base panel or at least one side panel of the one or more side panels forming the first compartment or the third compartment may include a plurality of perforations.

In a representative embodiment, the plurality of electrical interfaces may comprise at least one universal serial bus (USB) interface; and the hospital bed caddy may further comprise: a direct current (DC) power supply arranged in the second compartment and coupled to the USB port.

In a representative embodiment, the plurality of electrical interfaces comprise of one or more electrical interfaces selected from the group consisting of: a two-pronged alternating current (AC) electrical outlet, a three-pronged AC electrical outlet, a universal serial bus (USB) interface, a registered jack-11 (RJ-11) telecommunications network interface, a Deutsches Institut für Normung (DIN) connector or interface; and combinations thereof.

In a representative embodiment, bed rail attachment comprises: one or more hooks, or a removable adhesive, or one or more hook and loop fasteners.

In another representative embodiment, hospital bed caddy may further comprise: a pivot mount coupled to the housing; an extension arm coupled to the pivot mount; and a mobile device mount coupled to the extension arm.

In another representative embodiment, hospital bed caddy may further comprise: at least one lid rotatably coupled to a side panel of the one or more side panels of the plurality of compartments.

In a representative embodiment, the cable harness or sheath may comprises: a heat-shrinkable polymeric, tubular sheath; or one or more hook and loop fasteners; or an adhesive tape, or a wire arranged spirally around the plurality of electrical cables.

In another representative embodiment, the second compartment may be removably coupleable within the housing.

In another representative embodiment, a hospital bed caddy is provided, comprising: a housing comprising: a base panel having a first opening; and one or more side panels coupled to the base panel to form a plurality of compartments, the plurality of compartments comprising a first compartment and a second compartment; a bed rail attachment comprising a hook coupled to or integrally formed with the housing; a plurality of electrical interfaces arranged in the second compartment; a cover panel having one or more second openings, the cover panel coupled over the plurality of electrical interfaces to separate the second compartment from the first compartment; a plurality of electrical cables arranged in the first opening, each electrical cable of the plurality of electrical cables coupled within the second compartment to a corresponding electrical interface of the plurality of electrical interfaces; and a cable harness or sheath coupled around the plurality of electrical cables.

In another representative embodiment, a hospital bed caddy is provided, comprising: a housing comprising: a base panel having a first opening; and one or more side panels coupled to the base panel to form a plurality of compartments, the plurality of compartments comprising a first compartment, a second compartment, and a third compartment, wherein the base panel or at least one side panel of the one or more side panels forming the first compartment or the third compartment has a plurality of perforations, and wherein a portion of the base panel and at least one front side panel of the one or more side panels of the third compartment further comprise a slot or slot opening; a bed rail attachment comprising a hook coupled to or integrally formed with the housing; a plurality of electrical interfaces arranged in the second compartment, the plurality of electrical interfaces comprising at least one universal serial bus (USB) interface, wherein the plurality of electrical interfaces are user-accessible from the first compartment; a direct current (DC) power supply arranged in the second compartment and coupled to the USB port; a cover panel having one or more second openings, the cover panel coupled over the plurality of electrical interfaces to separate the second compartment from the first compartment; a pivot mount coupled to the housing; an extension arm coupled to the pivot mount; a mobile device mount coupled to the extension arm; a plurality of electrical cables arranged in the first opening, each electrical cable of the plurality of electrical cables coupled within the second compartment to a corresponding electrical interface of the plurality of electrical interfaces; and a cable harness or sheath coupled around the plurality of electrical cables.

Numerous other advantages and features of the present invention will become readily apparent from the following detailed description of the invention and the embodiments thereof, from the claims and from the accompanying drawings.

### BRIEF DESCRIPTION OF THE DRAWINGS

The objects, features and advantages of the present invention will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

FIG. 1 is an isometric view illustrating a representative first embodiment of a hospital bed caddy.

FIG. 2 is an isometric cut-away view illustrating the representative first embodiment of the hospital bed caddy.

FIG. 3 is a top, plan view illustrating the representative first embodiment of the hospital bed caddy.

FIG. 4 is a bottom view illustrating the representative first embodiment of the hospital bed caddy.

FIG. 5 is a representative electrical block diagram illustrating one or more electrical interfaces and electrical couplings of representative embodiments of the hospital bed caddy.

FIG. 6 is an isometric view illustrating a representative second embodiment of a hospital bed caddy.

FIG. 7 is an isometric view illustrating a representative third embodiment of a hospital bed caddy.

FIG. 8 is an isometric view illustrating a representative fourth embodiment of a hospital bed caddy.

FIG. 9 is a side view illustrating a second representative bed rail attachment of any of the various embodiments of the hospital bed caddy.

FIG. 10 is a side view illustrating a third representative bed rail attachment of any of the various embodiments of the hospital bed caddy.

FIG. 11 is a side view illustrating a fourth representative bed rail attachment of any of the various embodiments of the hospital bed caddy.

FIG. 12 is an isometric view illustrating a representative fifth embodiment of a hospital bed caddy.

FIG. 13 is an isometric view illustrating a representative sixth embodiment of a hospital bed caddy.

FIG. 14 is a front view illustrating the representative sixth embodiment of the hospital bed caddy.

FIG. 15 is a side view illustrating the representative sixth embodiment of the hospital bed caddy.

FIG. 16 is a top, plan view illustrating the representative sixth embodiment of the hospital bed caddy.

FIG. 17 is a bottom view illustrating the representative sixth embodiment of the hospital bed caddy.

### DETAILED DESCRIPTION OF REPRESENTATIVE EMBODIMENTS

While the present invention is susceptible of embodiment in many different forms, there are shown in the drawings and will be described herein in detail specific exemplary embodiments thereof, with the understanding that the present disclosure is to be considered as an exemplification of the principles of the invention and is not intended to limit the invention to the specific embodiments illustrated. In this respect, before explaining at least one embodiment consistent with the present invention in detail, it is to be understood that the invention is not limited in its application to the details of construction and to the arrangements of components set forth above and below, illustrated in the drawings, or as described in the examples. Methods and apparatuses consistent with the present invention are capable of other embodiments and of being practiced and carried out in various ways. Also, it is to be understood that the phraseology and terminology employed herein, as well as the abstract included below, are for the purposes of description and should not be regarded as limiting.

As mentioned above, various representative embodiments provide a hospital bed caddy having electrically active charging and communication interfaces with a mobile device mount and a cable harness. The representative embodiments improve the patient experience by facilitating organization and management of hospital and personal devices and cables in a hospital room. The representative embodiments provide for charging of mobile devices and for storage of such mobile devices, the patient remote control device, and other personal items, all within easy reach of the patient. The representative embodiments are also compatible with existing hospital infrastructures and avoid any potential interference with hospital devices and treatments.

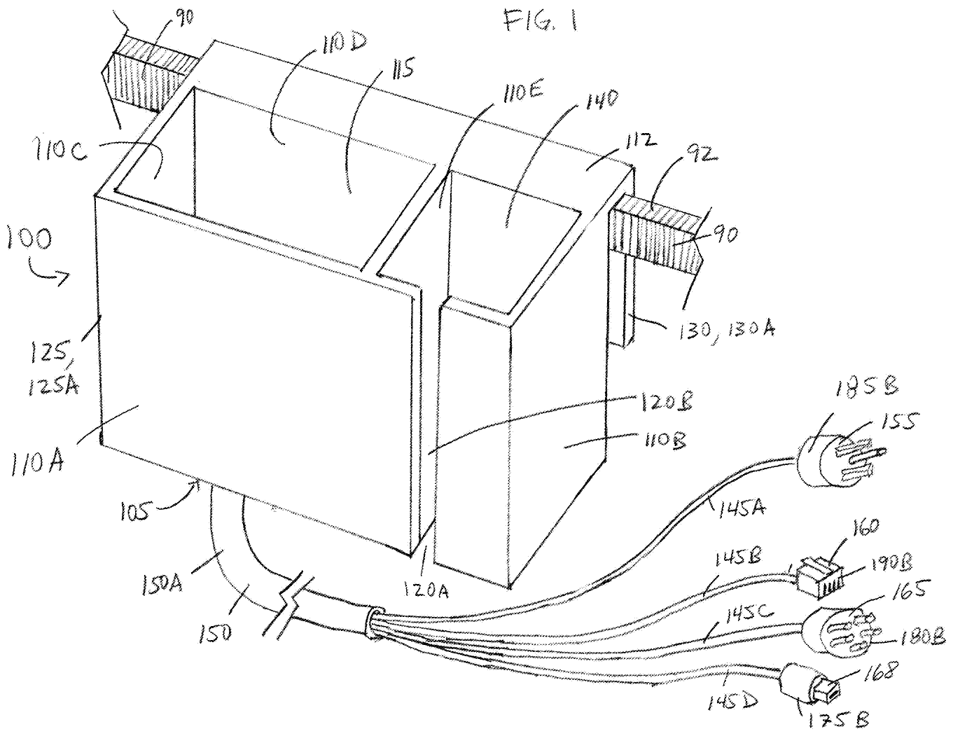

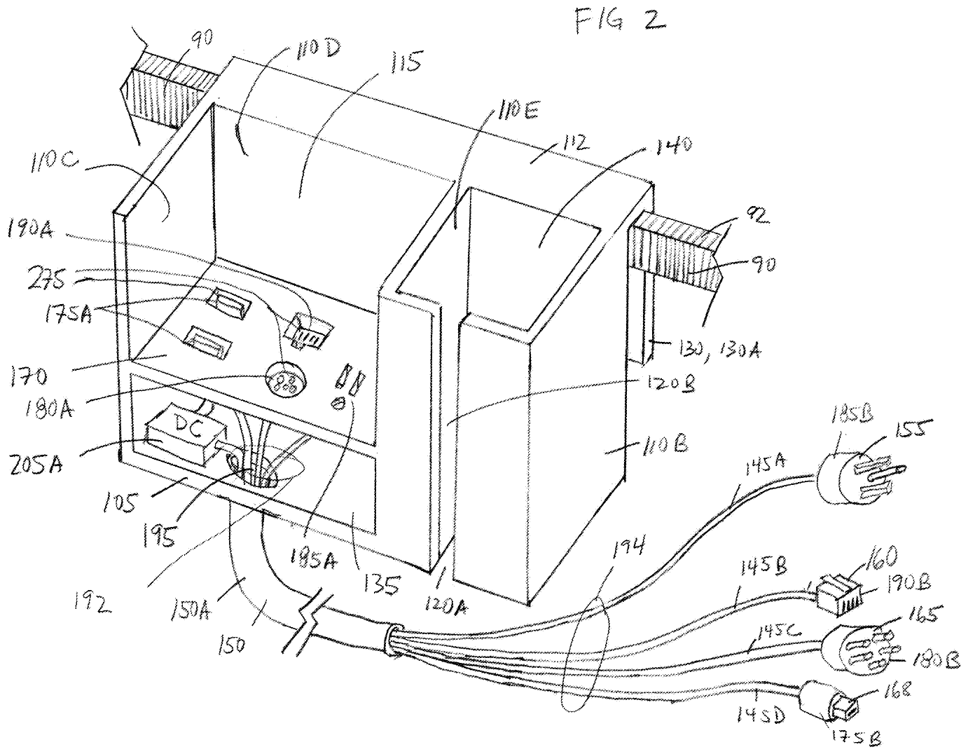

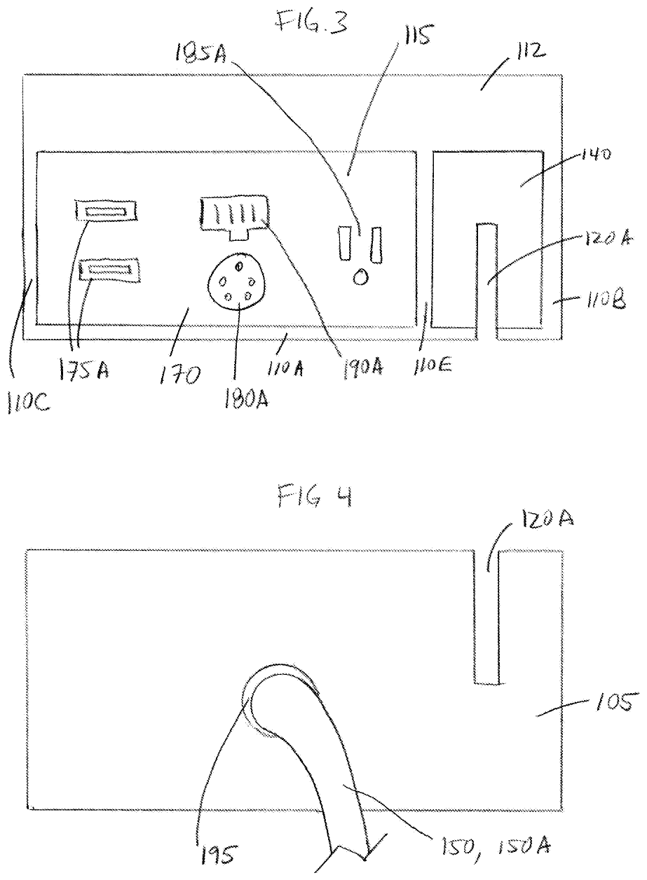

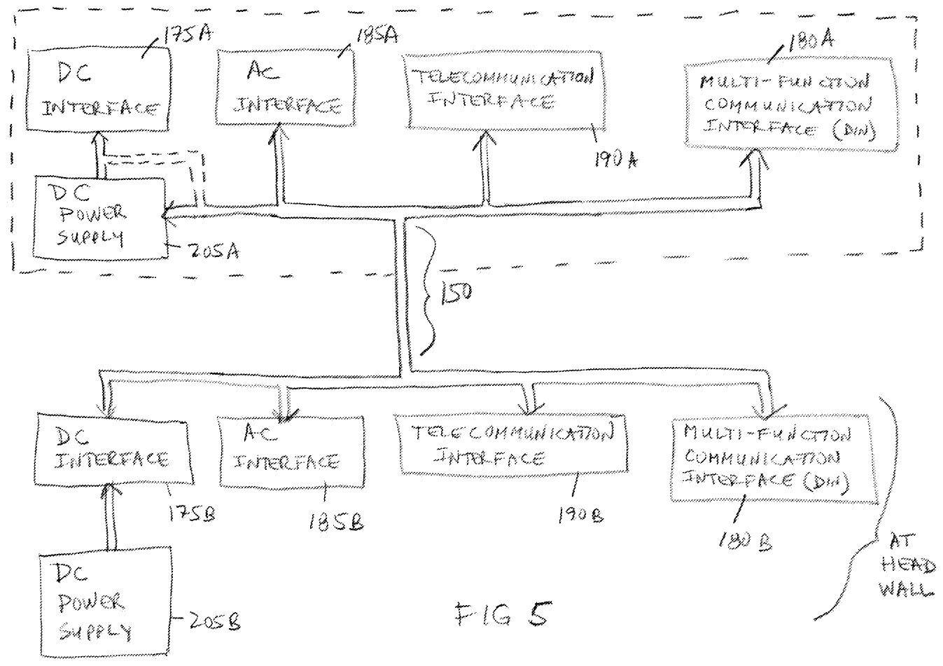

FIG. 1 is an isometric view illustrating a representative first embodiment of a hospital bed caddy 100. FIG. 2 is an isometric cut-away view illustrating the representative first embodiment of the hospital bed caddy 100. FIG. 3 is a top, plan view illustrating the representative first embodiment of the hospital bed caddy 100. FIG. 4 is a bottom view illustrating the representative first embodiment of the hospital bed caddy 100. FIG. 5 is a representative electrical block diagram illustrating one or more electrical interfaces and electrical couplings of representative embodiments of the hospital bed caddy 100, 200, 300, 400, 500, 600.

Referring to FIGS. 1-5, a representative first embodiment of a hospital bed caddy 100 comprises a housing 125, 125A formed by a base panel (or base) 105 coupled to one or more side panels (or side walls) 110, illustrated as a front side panel 110A, lateral side panels 110B and 110C, a rear (or back) side panel 110D, and an optional side (or divider) panel 110E. The side panels 110 coupled to the base panel 105 further form a plurality of compartments 115, 135, 140, illustrated as a first, main or central compartment 115, a second, electrical compartment 135, and an optional third, side compartment 140. One or more compartments 115, 140 of the plurality of compartments 115, 135, 140, may be utilized (by the patient) for organization and storage of a wide variety of possessions, devices, other items, etc., with the second, electrical compartment 135 utilized for housing one or more electrical interfaces (or, equivalently, connectors) 175A, 180A, 185A, 190A, as discussed in greater detail below. A cover panel (or plate) 170 forms a top panel or cover for the second, electrical compartment 135, and separates the second, electrical compartment 135 (with its electrical interfaces 175A, 180A, 185A, 190A) from the first compartment 115.

When the second, electrical compartment 135 is arranged horizontally (transversely) as illustrated in FIGS. 1-5 (with the horizontal (or transverse) direction being or defined with reference to an upper surface 92 of the hospital bed rail 90 (illustrated in FIGS. 1 and 2) when the hospital bed is flat (neither head nor foot of the bed raised or lowered), the cover panel 170 may also be considered to form a (second) base panel 105A of the first compartment 115. When the second, electrical compartment 135 is arranged vertically (longitudinally) (not separately illustrated), the cover panel 170 may also be considered to form a side panel 110 of the first compartment 115. Other configurations or locations for the second, electrical compartment 135 within the hospital bed caddy are discussed in greater detail below.

A bed rail attachment 130 (illustrated in FIG. 1 as a hook 130A) is coupled to or integrally formed with the housing 125, 125A. The bed rail attachment 130 has a form factor and size suitable to couple over or around (and hang from) a hospital bed rail 90 to secure the hospital bed caddy 100 to the hospital bed rail 90, while continuing to allow unrestricted movement of the hospital bed rail 90, as may be necessary or desirable. The hospital bed caddy 100 is typically positioned within reach of the patient in the hospital bed, and provides readily accessible and convenient storage and access to electrical charging and other functions, for a wide variety of devices. In addition to a hook 130A, other types or variations of bed rail attachments 130 are also available and within the scope of the disclosure, such as those illustrated and discussed below with reference to FIGS. 9-11.

The second, electrical compartment 135 includes one or more electrical interfaces (or, equivalently, connectors) 175A, 180A, 185A, 190A, illustrated as one or more direct current (DC) interfaces 175A (such as one or more Universal Serial Bus (USB) ports for DC charging, e.g., USB-A, USB-B, USB-C), a multi-function communication interface 180A (such as a 5-pin, 6-pin, or 8-pin (most common) DIN for coupling to a patient remote control device (not separately illustrated)), an optional AC power outlet (or interface) 185A for coupling to AC power, and a telecommunication interface 190A (such as an RJ-11 jack for coupling a landline telephone to landline communication lines). In a representative embodiment, the second, electrical compartment 135 also includes a DC power supply 205A coupled to the DC interface 175A to receive AC power and convert the AC to DC. In another representative embodiment, the second, electrical compartment 135 does not include a DC power supply, with a DC power supply 205B instead provided at the head wall, as discussed in greater detail below. Other types of electrical interfaces or connectors may be included, in addition to those illustrated and discussed herein, and any and all such electrical interfaces or connectors are within the scope of the disclosure.

The cover panel 170 has a plurality of (second) openings 275, each of which are sized and shaped correspondingly to match to each of the electrical interfaces 175A, 180A, 185A, 190A) which allow portions of each of the electrical interfaces 175A, 180A, 185A, 190A to be exposed and user-accessible within the first compartment 115 while the cover panel 170 nonetheless isolates the one or more electrical cables 145 and their attachments to the electrical interfaces 175A, 180A, 185A, 190A from the first compartment 115 (and the user). In operation, a hospital patient, care provider or other user may access the one or more electrical interfaces 175A, 180A, 185A, 190A of the second, electrical compartment 135 through the first compartment 115. For example, the user may plug a USB cable into one of the DC interfaces 175A to charge a mobile communication device (not separately illustrated), which in turn may be stored or held in the first compartment 115, within easy reach of the patient. Also for example, the user may plug a device charger into the AC interface 185A (such as an AC power outlet) to charge a computing device such as a laptop (not separately illustrated), which also in turn may be stored or held in the first compartment 115, also within easy reach of the patient. Continuing with the examples, the user may plug a telephone line into the telecommunications interface 190A (such as an RJ-11 jack) to connect a landline telephone (not separately illustrated), which also in turn may be stored or held in the first compartment 115, also within easy reach of the patient.

In other representative embodiments, not separately illustrated, the second, electrical compartment 135 may be arranged in other locations within any of the hospital bed caddies 100, 200, 300, 400, 500, 600. For example and without limitation, rather than the second, electrical compartment 135 being arranged horizontally with access to the electrical interfaces 175A, 180A, 185A, 190A provided at the bottom of the first compartment 115, the second, electrical compartment 135 may be arranged vertically with access to the electrical interfaces 175A, 180A, 185A, 190A provided at one or the sides of the first compartment 115. As another alternative, the second, electrical compartment 135 may be arranged horizontally between the first compartment 115 and the bed rail attachment 130, with access to the electrical interfaces 175A, 180A, 185A, 190A provided at the upper or top surface 112 of the hospital bed caddy 100, 200, 300, 400, 500, 600. As another alternative, the second, electrical compartment 135 may be arranged on one of the sides of the hospital bed caddy 100, 200, 300, 400, 500, 600, with access to the electrical interfaces 175A, 180A, 185A, 190A provided from an external surface of the hospital bed caddy 100, 200, 300, 400, 500, 600. Numerous other variations of the location of the second, electrical compartment 135 will be apparent to those having skill in the art, and any and all such variations are considered equivalent and within the scope of the disclosure.

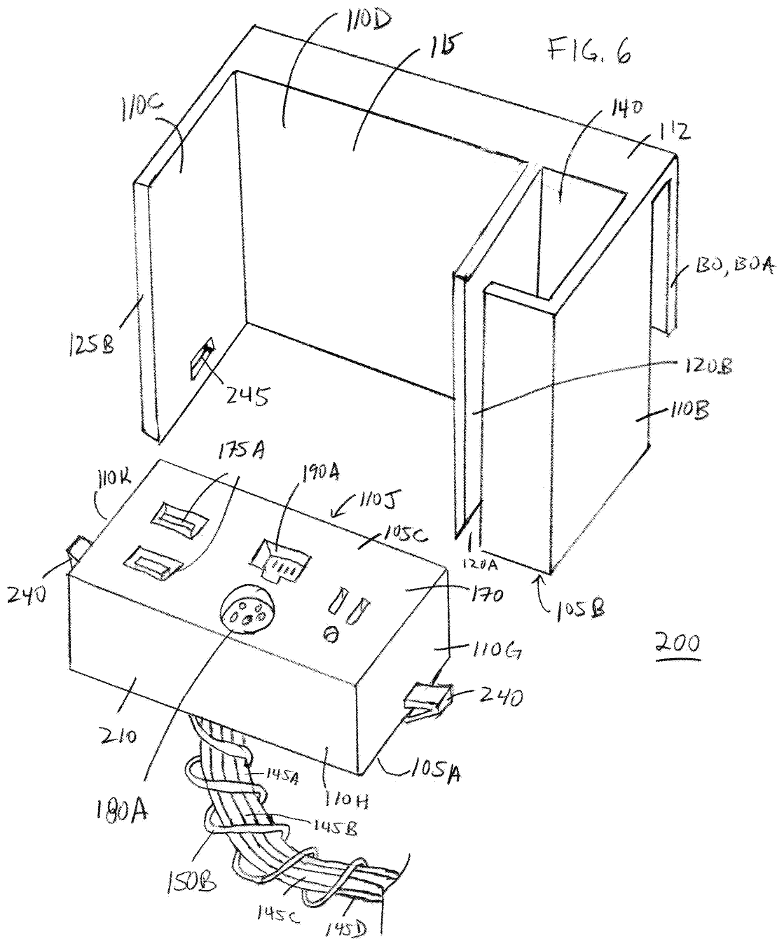

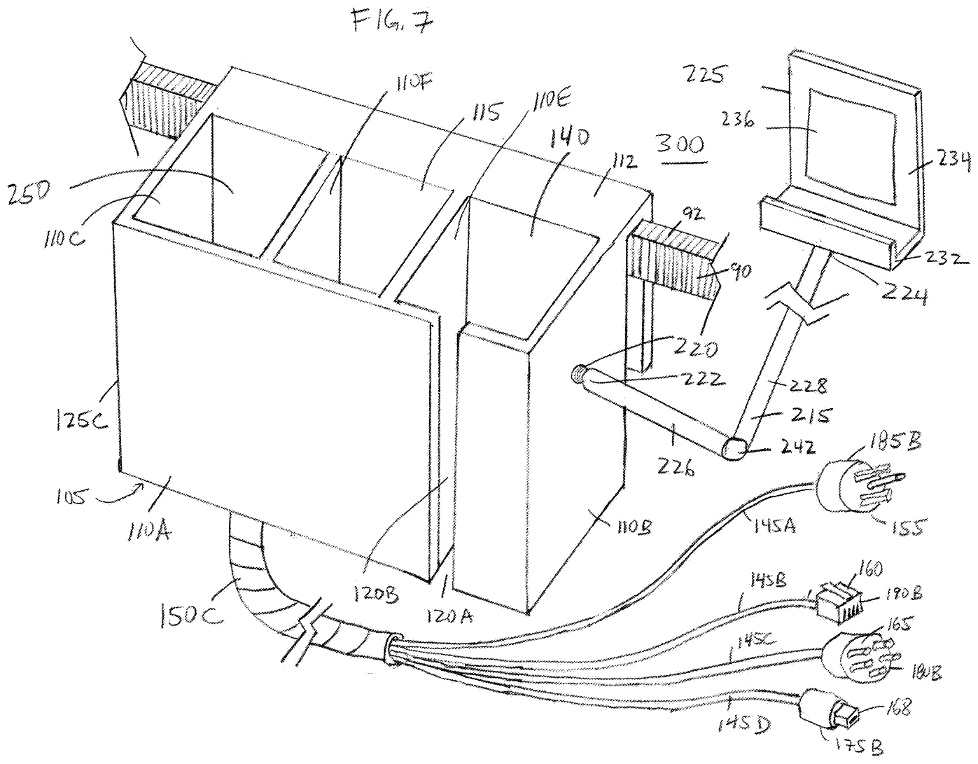

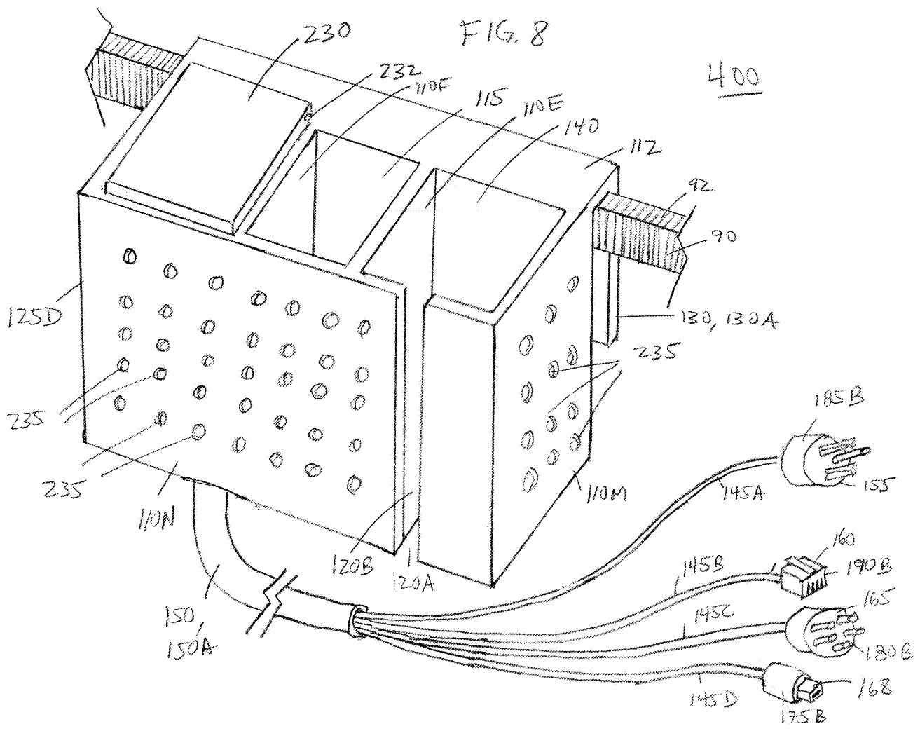

For any or all of these available configurations or locations of the second, electrical compartment 135, the base panel 105 typically includes a first opening 195 for passage of one or more electrical cables (wires) 145 into the second, electrical compartment 135. The electrical cables 145 are sized appropriately to extend between the housing 125 and the head wall. Generally, in the region between the housing 125 and the head wall, the one or more electrical cables 145 are held together collectively using a cable (or wire) harness or sheath 150, to appear and function as a single, unified cable, rather than having multiple cables which can become entangled or scattered. As illustrated in FIGS. 1-4, the cable (or wire) harness or sheath 150 is implemented as a polymeric, tubular sheath 150A, such as a polymeric sheath or wrap which is responsive to heat to shrink around and secure the electrical cables 145. Additional examples or types of cable (or wire) harnesses or sheaths 150 are illustrated and discussed below with reference to FIGS. 6 and 7, and all such variations are considered equivalent and within the scope of the disclosure.

Within the second, electrical compartment 135, the individual electrical cables 145 fan out (192) from the cable (or wire) harness or sheath 150 and are coupled to a corresponding electrical interface 175A, 180A, 185A, 190A, as illustrated in FIGS. 2 and 5. Similarly, at the head wall, the individual electrical cables 145 also fan out (194) from the cable (or wire) harness or sheath 150 and are coupled to counterpart electrical interfaces 175B, 180B, 185B, 190B, for coupling (e.g., plugging in) to corresponding electrical interfaces (connectors or outlets) arranged on or in the head wall. For example, the AC power outlet (or interface) 185A (and/or DC power supply 205) is coupled through an electrical cable 145A to a counterpart AC electrical interface 185B, illustrated for example in FIGS. 1 and 2 as an AC plug 155, to be inserted into an AC outlet in or on the head wall. Also for example, the telecommunication interface 190A (such as an RJ-11 jack) is coupled through an electrical cable 145B to a counterpart electrical telecommunication interface 190B, illustrated for example in FIGS. 1 and 2 as an RJ-11 connector 160, which in turn is to be inserted into an RJ-11 jack in or on the head wall.

As mentioned above, a DC power supply 205B may be provided at the head wall instead of including a DC power supply 205A in the second, electrical compartment 135. For this embodiment, the DC power interface 175A (such as a female USB port) is coupled through an electrical cable 145D to a counterpart DC electrical interface 175B, illustrated for example in FIGS. 1 and 2 as a male USB connector 168, for example, which in turn would be inserted into a female USB port coupled to a DC power supply 205B (typically coupled to AC power) in or on the head wall. When a DC power supply 205A is included in the second, electrical compartment 135, it may receive AC power via the electrical cable 145A (coupleable to AC power).

In a representative embodiment, a multi-function communication interface 180A is also provided in the second, electrical compartment 135 for coupling to a patient remote control device, discussed above, which provides a wide variety of functions, including providing an alert to the nurses' station (such as to summon a nurse or other care provider), providing a microphone and speaker for communication with the nurse or other care provider, providing controls for a television in the hospital room (such as power on/off, volume and channel selection), and may also provide control over positioning of portions of the bed (such as raising and lowering the head or the foot of the bed)). For this example, the multi-function communication interface 180A (such as a female DIN interface connector) is coupled through an electrical cable 145C to a counterpart electrical interface 180B, illustrated in FIGS. 1 and 2 as a male DIN connector 165, to be inserted into a female DIN connector in or on the head wall.

The various electrical interfaces 175A, 180A, 185A, 190A, 175B, 180B, 185B, 190B, DC power supply 205A, 205B, and electrical cables 145 may be implemented as known or becomes known in the electronics field.

In a representative embodiment, the base panel 105 and the front panel 110A (or another side panel 110) may include additional openings, illustrated as an opening 120A configured as a first slot or slot opening 120A in the base panel 105, which is contiguous and in communication with another opening 120B configured as a second slot or slot opening 120B formed in the front panel 110A (or another side panel 110) (collectively forming a slot or slot opening 120). The slot 120 is configured to allow passage of a communication cable, such as a cable from a patient remote control device. For example, rather than utilizing the multi-function communication interface 180, as an option (not separately illustrated), the patient remote control device may remain coupled through a corresponding communication cable directly to a corresponding interface arranged in the head wall of the hospital room, such as a corresponding DIN connector. For this embodiment, the communication cable of the patient remote control device may slide through the slot 120, so that the patient remote control device may be inserted and cradled (and thereby stored), for example, in the third compartment 140, within easy reach of the patient, and avoiding any need for attempting to tether the patient remote control device by wrapping its cable around the hospital bed rail, also for example.

It should be noted that a magnetic charging interface is generally not included within the hospital bed caddy 100, 200, 300, 400, 500, 600. First, to actually charge a device, typical magnetic charging interfaces need to be tightly coupled to and aligned with the device, with the device not merely placed in the vicinity of the magnetic charging interface. As a result, it is unlikely that a magnetic charging interface will actually work to charge a mobile device within the caddy, absent other structures to correctly position and hold the specific type of mobile device tightly against such a magnetic charging interface. Second, magnetic charging potentially could create other concerns; for example, fringing fields from the magnetic charging interface could potentially interfere with nearby medical equipment and patient monitors, including wireless transmission of EKG data, for example and without limitation.

FIG. 6 is an isometric view illustrating a representative second embodiment of a hospital bed caddy 200. The hospital bed caddy 200 differs from the hospital bed caddy 100 insofar as the second, electrical compartment 135 is provided as an electrical chassis 210 removably coupleable to the housing 125B, using any appropriate fastener, such as via the illustrated latches 240 (or, equivalently, tabs or detents) of the electrical chassis 210 which are removably insertable into corresponding openings 245 of the base panel 105A and/or lateral side panels 110C, 110E of the housing 125B. Other types of fasteners (280) are also discussed below. The electrical chassis 210 has its own (second) base 105B and side panels 110G, 110H, 110J, and 110K, and includes the various electrical interfaces 175A, 180A, 185A, 190A discussed above, and similarly is coupled through the electrical cables 145 having the cable (or wire) harness or sheath 150 to the head wall. One advantage to this second embodiment is that the electrical chassis 210 may be sanitized and re-used, while the balance of the hospital bed caddy 200 may be disposed of and replaced with a new housing 125B, such as for use with a next patient in the hospital room. The hospital bed caddy 200 further differs from the hospital bed caddy 100 insofar as the cable (or wire) harness or sheath 150B is implemented as a flexible wire, cable, or cord wrapped in a spiral around the electrical cables 145. In this configuration, the cover panel 170 functions to form a (third) base panel 105C of the first compartment 115. Apart from these variations, the hospital bed caddy 200 has the same electrical and storage functionality as the hospital bed caddy 100 and may be utilized equivalently or interchangeably.

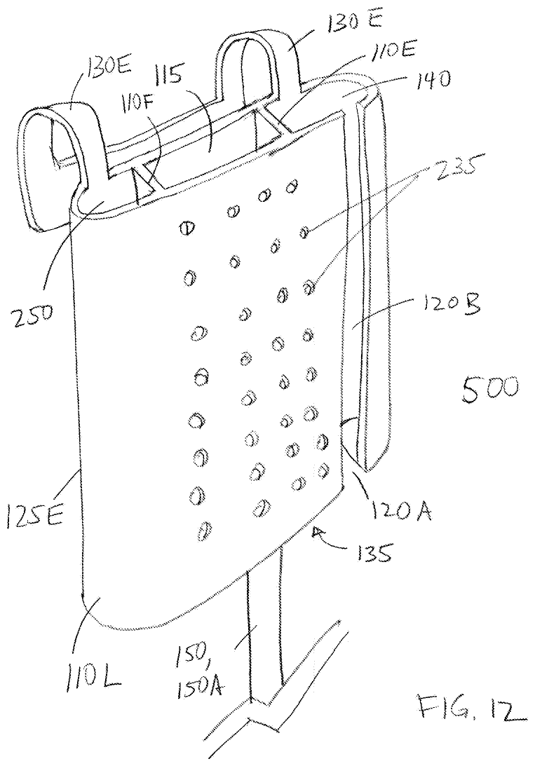

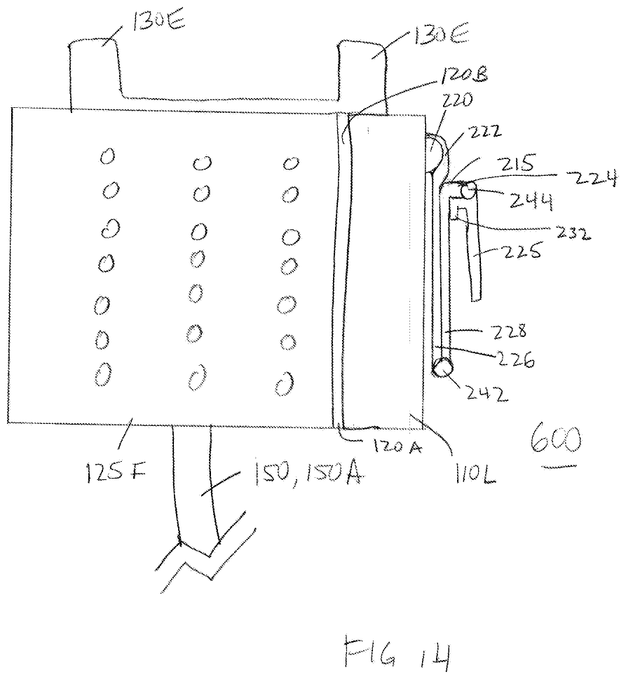

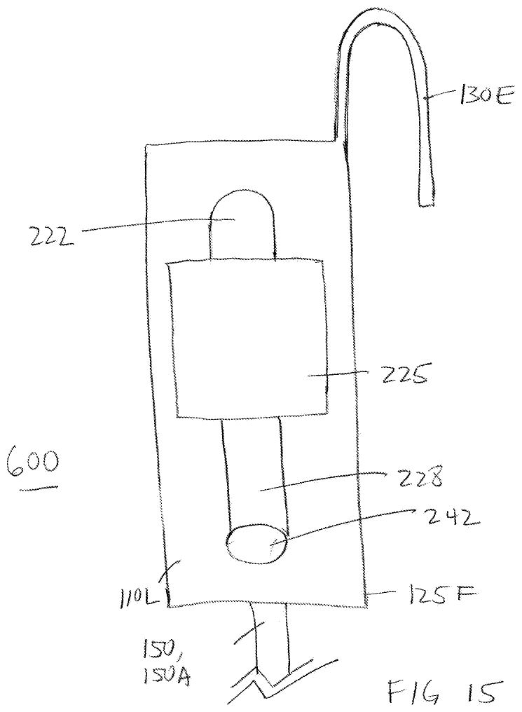

FIG. 7 is an isometric view illustrating a representative third embodiment of a hospital bed caddy 300. The hospital bed caddy 300 differs from the hospital bed caddy 100 insofar as the hospital bed caddy 300 includes an optional fourth compartment 250 formed using an additional side (or divider) panel 110F, and further comprises an extension arm 215 with a mobile device mount 225, with the extension arm 215 illustrated in an extended position in FIG. 7. The extension arm 215 has multiple, jointed (or hinged) segments 226, 228 as illustrated (using joints or hinges 242, 244, along with the pivot coupling or mount 220), to allow for compact folding of the extension arm (as illustrated in FIG. 11) and extending of the extension arm 215 (as illustrated in FIGS. 7 and 13) to reach from the housing 125C to a patient seated or lying in the hospital bed. Any suitable joints or hinges 242, 244 may be utilized for the extension arm 215. A first end 222 of the extension arm 215 is coupled to the housing 125 using a pivot mount or coupling 220 (such as a ball and socket coupling), to allow the extension arm 215 to have a wide range of motion around or from the housing 125C. A second end 224 of the extension arm 215 is coupled to a mobile device mount 225, typically also using a rotatable or pivotable mount, coupling or hinge 244 (illustrated in FIG. 14) which can hold and secure a mobile device (such as a mobile telephone or tablet) and, through the movement of the extension arm 215, allow a mobile device to be held and secured in a suitable position selected by the user, such as the patient. The hospital bed caddy 300 further differs from the hospital bed caddy 100 insofar as the cable (or wire) harness or sheath 150C is implemented as a tape wrapped in a spiral around the electrical cables 145, such as an adhesive tape which may adhere to and secure the electrical cables 145, or as a hook and loop cable tie which also may wrap around and secure the electrical cables 145, for example and without limitation. Apart from these variations, the hospital bed caddy 300 has the same electrical and storage functionality as the hospital bed caddy 100 and may be utilized equivalently or interchangeably.

The extension arm 215 may have more or fewer segments than those illustrated, and is generally sized, when extended, to reach a patient lying or seated in the hospital bed. In addition, those having skill in the art will recognize that the extension arm 215 may be implemented in a wide variety of ways, such as by using a flexible (or bendable) support arm or gooseneck (e.g., a bendable wire) without a pivot mount or coupling 220, a telescoping arm, any other type of folding or scissor arm, and all such variations are considered equivalent and within the scope of the disclosure.

The mobile device mount 225 may be implemented in a wide variety of ways. In the illustrated embodiment, the mobile device mount 225 is implemented as a generally flat panel 234 having a lip (or ledge) 232, with a mobile device placed against the flat panel 234 and rested on top of the lip (or ledge) 232. In lieu of or in addition to the lip (or ledge) 232, the flat panel 234 of the mobile device mount 225 may include a removable adhesive 236 (which adhesive may be implemented as known in the art), to which the mobile device may be adhered and easily removed. Such a removable adhesive 236 may also be removed from the flat panel 234 and replaced, as needed, such as for a new occupant of the hospital room. The mobile device mount 225 may also be implemented with a magnetic coupling, as known in the art, and not separately illustrated.

The extension arm 215 and mobile device mount 225 function to hold a mobile device at a desired distance from the patient while the patient is lying or seated in the hospital bed, allowing the patient to utilize the mobile device comfortably. Those having skill in the art will recognize that the extension arm 215 may be coupled to the housing 125 in any of a wide variety of locations, all of which are considered equivalent and within the scope of the disclosure. Examples of structures of the extension arm 215 and the mobile device mount 225 that may be utilized may include the Innovative Tablik iPad, Tablet Arm Desk Mount (http://www.monitordeskmount.com/monitor-mounts/innovative-tablik-ipad-tablet-arm-desk-mount-p-1263.html), the CTA Tabletop Arm Mount (https://www.cdw.com/product/cta-tabletop-arm-mount-mounting-kit-for-tablet/6318861), and another adjustable arm and holder available at https://alexnld.com/product/ap-7lc-360-degree-rotating-adjustable-lifting-long-arm-lazy-bedside-desktop-tablets-phones-holder. Numerous other variations of the extension arm 215 and the mobile device mount 225 will be apparent to those having skill in the art, and any and all such variations are considered equivalent and within the scope of the disclosure.

FIG. 8 is an isometric view illustrating a representative fourth embodiment of a hospital bed caddy 400. The hospital bed caddy 400 also differs from the hospital bed caddy 100 insofar as the hospital bed caddy 400 includes an optional fourth compartment 250 formed using an additional side (or divider) panel 110F. The hospital bed caddy 400 further differs from the hospital bed caddy 100 insofar as: (1) one or more lids 230 are provided to cover a corresponding compartment (e.g., covering compartment 250), with a representative lid 230 rotatably coupled to the housing 125D using a hinge 232; and (2) one or more of the base panel 105 (not separately illustrated) and/or side panels 110M, 110N further include a plurality of perforations (or holes) 235, such as to provide additional or greater airflow within one or more of the compartments 115, 140, 250. These perforations (or holes) 235 may be helpful in dissipating any condensation which may form around contents held in the hospital bed caddy 400, such as a bottle, pitcher, cup or other container having cold water, for example and without limitation. Apart from these variations, the hospital bed caddy 400 has the same electrical and storage functionality as the hospital bed caddy 100 and may be utilized equivalently or interchangeably.

FIG. 9 is a side view illustrating a second representative bed rail attachment 130B which may be utilized with or as part of any of the various embodiments of the hospital bed caddy 100, 200, 300, 400, 500, 600. As illustrated, the bed rail attachment 130B comprises a generally flat housing extension 260 (which may be coupled to or integrally formed with the housing 125) which can be placed on top of a hospital bed rail 90, and held in place using one or more hook and loop attachments 265 (e.g., Velcro) wrapped around the hospital bed rail 90, illustrated as a loop attachment 265A and corresponding hook attachments 265B, with the hook attachments 265B adhered to the housing 125 and housing extension 260.

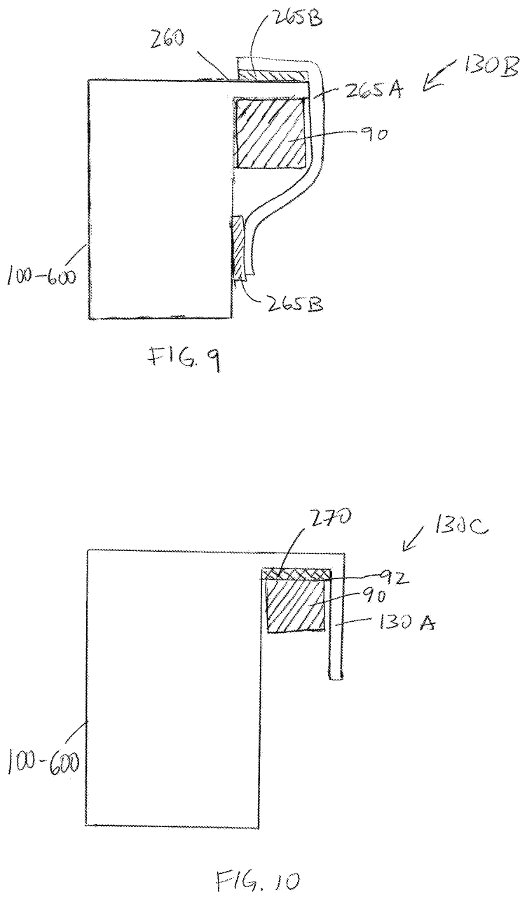

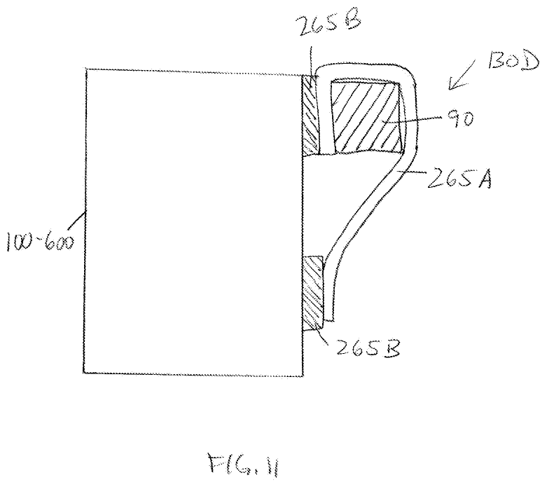

FIG. 10 is a side view illustrating a third representative bed rail attachment 130C which may be utilized with or as part of any of the various embodiments of the hospital bed caddy 100, 200, 300, 400, 500, 600. As illustrated, the bed rail attachment 130C is also implemented as a hook 130A which wraps around the hospital bed rail 90, but further comprises an adhesive 270 arranged between the hook 130A and the top or upper surface 92 of the hospital bed rail 90, such as a removeable adhesive.

FIG. 11 is a side view illustrating a fourth representative bed rail attachment 130D which may be utilized with or as part of any of the various embodiments of the hospital bed caddy 100, 200, 300, 400, 500, 600. As illustrated, the bed rail attachment 130D comprises one or more hook and loop attachments 265 (e.g., Velcro) wrapped around the hospital bed rail 90, illustrated as a loop attachment 265A and corresponding hook attachments 265B, with the hook attachments 265B adhered to the housing 125. In addition to those illustrated in the various Figures, numerous other variations of the configuration and possible locations of the bed rail attachment 130 will be apparent to those having skill in the art, and any and all such variations are considered equivalent and within the scope of the disclosure.

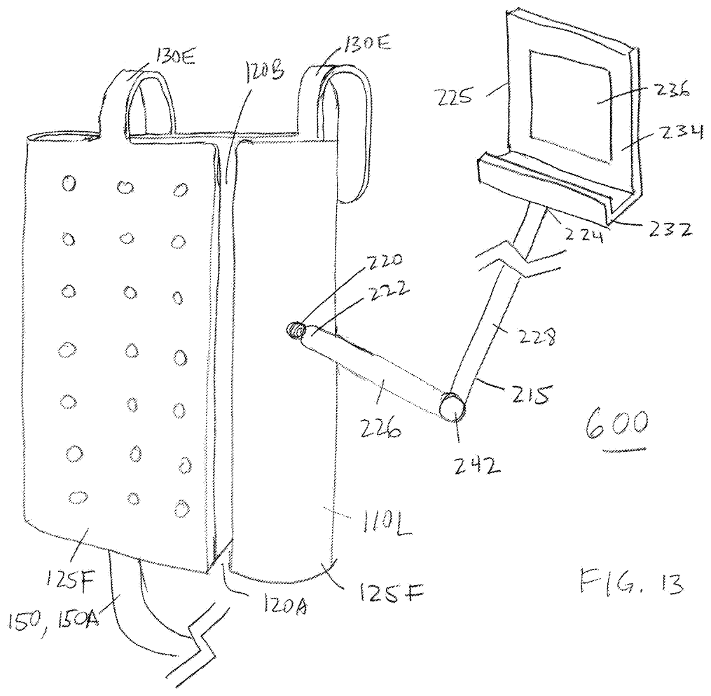

FIG. 12 is an isometric view illustrating a representative fifth embodiment of a hospital bed caddy 500. The hospital bed caddy 500 also differs from the hospital bed caddy 100 insofar as the hospital bed caddy 500 includes an optional fourth compartment 250 formed using an additional side (or divider) panel 110F. The hospital bed caddy 500 further differs from the hospital bed caddy 100 insofar as one or more of the base panel 105 and/or front side panel 110A further include a plurality of perforations (or holes) 235, also such as to provide additional or greater airflow within one or more of the compartments 115, 140, 250. The hospital bed caddy 500 further differs from the hospital bed caddy 100 insofar as the hospital bed caddy 500 has side panels 110 which are both flat and curvilinear as illustrated, and having a bed rail attachment 130 also configured as two joined or connected hooks 130E, providing a significant aesthetic variation for the hospital bed caddy 500. Apart from these variations, the hospital bed caddy 500 has the same electrical and storage functionality as the hospital bed caddy 100 and may be utilized equivalently or interchangeably.

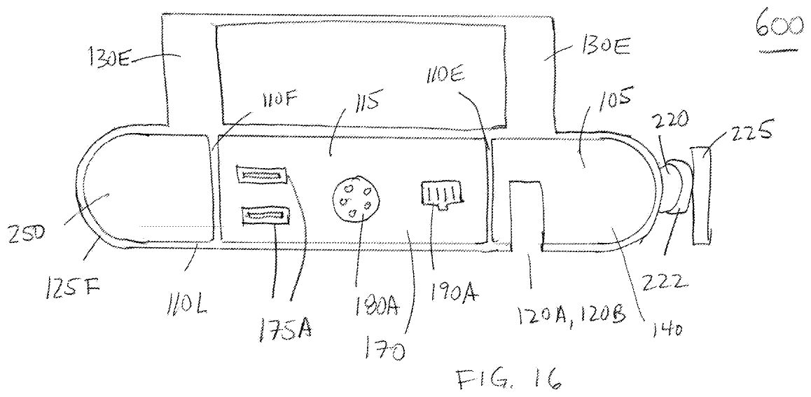

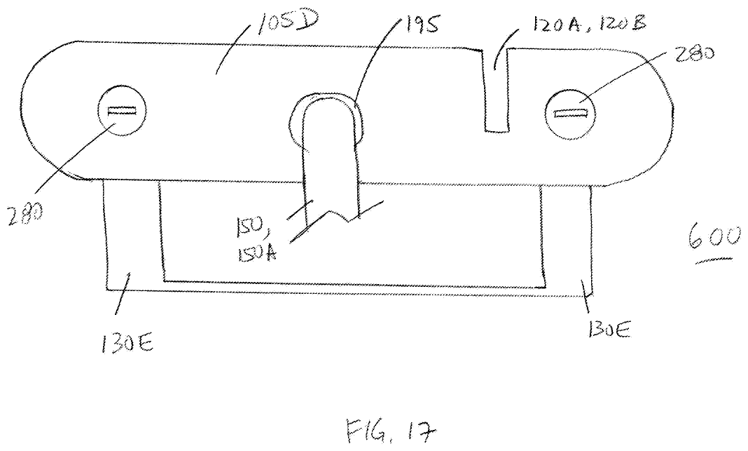

FIG. 13 is an isometric view illustrating a representative sixth embodiment of a hospital bed caddy 600. FIG. 14 is a front view illustrating the representative sixth embodiment of the hospital bed caddy 600. FIG. 15 is a side view illustrating the representative sixth embodiment of the hospital bed caddy 600. FIG. 16 is a top, plan view illustrating the representative sixth embodiment of the hospital bed caddy 600. FIG. 17 is a bottom view illustrating the representative sixth embodiment of the hospital bed caddy 600.

Referring to FIGS. 13-17, the hospital bed caddy 600 differs from the hospital bed caddy 500 insofar as the hospital bed caddy 600 also includes the extension arm 215 having the pivot coupling 220 with a mobile device mount 225, as discussed previously with reference to FIG. 7. The extension arm 215 is illustrated in the extended position in FIG. 13 and in a retracted position (folded and placed against the side panel 110L) in FIGS. 14 and 15. The hospital bed caddy 600 also differs from the hospital bed caddies 100, 500 insofar as the hospital bed caddy 600 does not include an AC electrical interface 185A. The hospital bed caddy 600 also differs from the hospital bed caddy 100 insofar as the hospital bed caddy 600 includes an optional fourth compartment 250 formed using an additional side (or divider) panel 110F. The hospital bed caddy 600 further differs from the hospital bed caddy 100 insofar as one or more of the base panel 105 and/or front side panel 110A further include a plurality of perforations (or holes) 235, also such as to provide additional or greater airflow within one or more of the compartments 115, 140, 250. The hospital bed caddy 600 further differs from the hospital bed caddy 100 insofar as the hospital bed caddy 600 has side panels 110 which are both flat (110E, 110F) and curvilinear (110L) as illustrated, and having a bed rail attachment 130 also configured as two joined or connected hooks 130E, providing a significant aesthetic variation for the hospital bed caddy 500. FIG. 17 also illustrates coupling of the base 105 to the rest of the housing 125F, using fasteners 280, such as screws, for example and without limitation. For example, the one or more electrical interfaces (or, equivalently, connectors) 175A, 180A, 185A, 190A may be inserted into the second, electrical compartment 135, coupled to corresponding electrical cables 145, followed by attachment of the base 105D using the fasteners 280. Apart from these variations, the hospital bed caddy 600 has the same DC electrical and storage functionality as the hospital bed caddy 100 and may be utilized equivalently or interchangeably.

As mentioned above, the representative embodiments of the hospital bed caddy 100, 200, 300, 400, 500, 600, and any of its various components such as compartments 115, 135, 140, 250, the bed rail attachment 130, etc., may have any size (height, width, depth), shape, or form factor suitable for use with a hospital bed and for coupling to a hospital bed rail 90, in addition to those illustrated, and all such variations are considered equivalent and within the scope of the disclosure. In addition, the representative embodiments of the hospital bed caddy 100, 200, 300, 400, 500, 600 illustrate different combinations of features and elements, with any and all mixing and matching of any of the various features and elements and any and all combinations of any of the various features and elements are within the scope hereof.

The housing 125, including the various side panels 110 and the base panel 105, along with the bed rail attachment 130, the cover panel 170, the lid 230, and other components may be fabricated in a wide variety of ways, including integrally formed (e.g., injection molded, 3D printed) or assembled from separate components (e.g., using any suitable fasteners or adhesives, not separately illustrated), and all such variations are considered equivalent and within the scope of the disclosure. The housing 125, including the various side panels 110 and the base panel 105, along with the bed rail attachment 130, the cover panel 170, the lid 230, and other components, may be implemented using any suitable material, and may be opaque or transparent, with suitable materials including any rigid (or semi-flexible) polymer or plastic, such as polyvinylchloride (PVC), polystyrene, polyacrylate, polytetrafluoroethylene (PTFE or Teflon), nylon, polycarbonates, polyesters, carbon fiber, glass, wood, particle board, silicone, silicone rubber, a metal, an alloy, etc., for example and without limitation, and all such variations are considered equivalent and within the scope of the disclosure. The housing 125, including the various side panels 110 and the base panel 105, along with the bed rail attachment 130, the cover panel 170, the lid 230, and other components also may have one or more coatings (not separately illustrated), such as an antibiotic or antimicrobial coating, for example and without limitation.

Representative examples of suitable polymers include, but are not limited to, fluorinated polymers or copolymers such as poly(vinylidene fluoride), poly(vinylidene fluoride-co-hexafluoropropene), poly(tetrafluoroethylene), and expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D,L-lactic acid); poly(D,L-lactide); poly(glycolic acid-cotrimethylene carbonate); poly(phosphoesters); poly(phosphoester urethane); poly(trimethylene carbonate); poly(iminocarbonate); poly(ethylene); and any derivatives, analogs, homologues, congeners, salts, copolymers and combinations thereof.

The polymers may also include, but are not limited to, poly(propylene) co-poly(ether-esters) such as, for example, poly(dioxanone) and poly(ethylene oxide)/poly(lactic acid); poly(anhydrides), poly(alkylene oxalates); poly(phosphazenes); poly(urethanes); silicones; silicone rubber; poly(esters); poly(olefins); copolymers of poly(isobutylene); copolymers of ethylene-alphaolefin; vinyl halide polymers and copolymers such as poly(vinyl chloride); poly(vinyl ethers) such as, for example, poly(vinyl methyl ether); poly(vinylidene halides) such as, for example, poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins such as poly(ethylene-co-vinyl alcohol) (EVAL), copolymers of acrylonitrile-styrene, ABS resins, and copolymers of ethylene-vinyl acetate; and any derivatives, analogs, homologues, congeners, salts, copolymers and combinations thereof.

The polymers may further include, but are not limited to, poly(amides) such as Nylon 66 and poly(caprolactam); alkyd resins; poly(carbonates); poly(oxymethylenes); poly(imides); poly(ester amides); poly(ethers) including poly(alkylene glycols) such as, for example, poly(ethylene glycol) and poly(propylene glycol); epoxy resins; polyurethanes; rayon; rayon-triacetate; biomolecules such as, for example, fibrin, fibrinogen, starch, poly(amino acids); peptides, proteins, gelatin, chondroitin sulfate, dermatan sulfate (a copolymer of D-glucuronic acid or L-iduronic acid and N-acetyl-D-galactosamine), collagen, hyaluronic acid, and glycosaminoglycans; other polysaccharides such as, for example, poly(N-acetylglucosamine), chitin, chitosan, cellulose, cellulose acetate, cellulose butyrate, cellulose acetate butyrate, cellophane, cellulose nitrate, cellulose propionate, cellulose ethers, and carboxymethylcellulose; and any derivatives, analogs, homologues, congeners, salts, copolymers and combinations thereof.

The present disclosure is to be considered as an exemplification of the principles of the invention and is not intended to limit the invention to the specific embodiments illustrated. In this respect, it is to be understood that the invention is not limited in its application to the details of construction and to the arrangements of components set forth above and below, illustrated in the drawings, or as described in the examples. Systems, methods and apparatuses consistent with the present invention are capable of other embodiments and of being practiced and carried out in various ways.

Although the invention has been described with respect to specific embodiments thereof, these embodiments are merely illustrative and not restrictive of the invention. In the description herein, numerous specific details are provided, such as examples of electronic components, electronic and structural connections, materials, and structural variations, to provide a thorough understanding of embodiments of the present invention. One skilled in the relevant art will recognize, however, that an embodiment of the invention can be practiced without one or more of the specific details, or with other apparatus, systems, assemblies, components, materials, parts, etc. In other instances, well-known structures, materials, or operations are not specifically shown or described in detail to avoid obscuring aspects of embodiments of the present invention. In addition, the various Figures are not drawn to scale and should not be regarded as limiting.

Reference throughout this specification to “one embodiment”, “an embodiment”, or a specific “embodiment” means that a particular feature, structure, or characteristic described in connection with the embodiment is included in at least one embodiment of the present invention and not necessarily in all embodiments, and further, are not necessarily referring to the same embodiment. Furthermore, the particular features, structures, or characteristics of any specific embodiment of the present invention may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

It will also be appreciated that one or more of the elements depicted in the Figures can also be implemented in a more separate or integrated manner, or even removed or rendered inoperable in certain cases, as may be useful in accordance with a particular application. Integrally formed combinations of components are also within the scope of the invention, particularly for embodiments in which a separation or combination of discrete components is unclear or indiscernible. In addition, use of the term “coupled” herein, including in its various forms such as “coupling” or “couplable”, means and includes any direct or indirect electrical or structural coupling, connection or attachment, or adaptation or capability for such a direct or indirect electrical or structural coupling, connection or attachment, including integrally formed components and components which are coupled via or through another component.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. In addition, every intervening sub-range within range is contemplated, in any combination, and is within the scope of the disclosure. For example, for the range of 5-10, the sub-ranges 5-6, 5-7, 5-8, 5-9, 6-7, 6-8, 6-9, 6-10, 7-8, 7-9, 7-10, 8-9, 8-10, and 9-10 are contemplated and within the scope of the disclosed range.

Furthermore, any signal arrows in the drawings/Figures should be considered only exemplary, and not limiting, unless otherwise specifically noted. Combinations of components of steps will also be considered within the scope of the present invention, particularly where the ability to separate or combine is unclear or foreseeable. The disjunctive term “or”, as used herein and throughout the claims that follow, is generally intended to mean “and/or”, having both conjunctive and disjunctive meanings (and is not confined to an “exclusive or” meaning), unless otherwise indicated. As used in the description herein and throughout the claims that follow, “a”, “an”, and “the” include plural references unless the context clearly dictates otherwise. Also as used in the description herein and throughout the claims that follow, the meaning of “in” includes “in” and “on” unless the context clearly dictates otherwise.

The foregoing description of illustrated embodiments of the present invention, including what is described in the summary or in the abstract, is not intended to be exhaustive or to limit the invention to the precise forms disclosed herein. From the foregoing, it will be observed that numerous variations, modifications and substitutions are intended and may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

It is claimed:

1. A hospital bed caddy, comprising:
a housing comprising:
a base panel having a first opening; and
one or more side panels coupled to the base panel to form a plurality of compartments, the plurality of compartments comprising a first compartment and a second compartment;
a bed rail attachment coupled to or integrally formed with the housing;
a plurality of electrical interfaces arranged in the second compartment;
a plurality of electrical cables arranged in the first opening, each electrical cable of the plurality of electrical cables coupled within the second compartment to a corresponding electrical interface of the plurality of electrical interfaces; and
a cable harness or sheath coupled around the plurality of electrical cables.

2. The hospital bed caddy of claim 1, further comprising:
a cover panel having one or more second openings, the cover panel coupled over the plurality of electrical interfaces to separate the second compartment from the first compartment.

3. The hospital bed caddy of claim 2, wherein the plurality of electrical interfaces are user-accessible from within the first compartment.

4. The hospital bed caddy of claim 1, wherein the base panel and the one or more side panels further form a third compartment, of the plurality of compartments, the third compartment arranged adjacent to the first compartment.

5. The hospital bed caddy of claim 4, wherein a portion of the base panel and at least one front side panel of the one or more side panels of the third compartment further comprise a slot or slot opening.

6. The hospital bed caddy of claim 1, wherein the base panel or at least one side panel of the one or more side panels forming the first compartment or the third compartment has a plurality of perforations.

7. The hospital bed caddy of claim 1, wherein the plurality of electrical interfaces comprise at least one universal serial bus (USB) interface; and wherein the hospital bed caddy further comprises:
a direct current (DC) power supply arranged in the second compartment and coupled to the USB port.

8. The hospital bed caddy of claim 1, wherein the plurality of electrical interfaces comprise of one or more electrical interfaces selected from the group consisting of: a two-pronged alternating current (AC) electrical outlet, a three-pronged AC electrical outlet, a universal serial bus (USB) interface, a registered jack-11 (RJ-11) telecommunications network interface, a Deutsches Institut für Normung (DIN) connector or interface; and combinations thereof.

9. The hospital bed caddy of claim 1, wherein the bed rail attachment comprises:
one or more hooks, or a removable adhesive, or one or more hook and loop fasteners.

10. The hospital bed caddy of claim 1, further comprising:
a pivot mount coupled to the housing;
an extension arm coupled to the pivot mount; and
a mobile device mount coupled to the extension arm.

11. The hospital bed caddy of claim 1, further comprising:
at least one lid rotatably coupled to a side panel of the one or more side panels of the plurality of compartments.

12. The hospital bed caddy of claim 1, wherein the cable harness or sheath comprises:
a heat-shrinkable polymeric, tubular sheath; or one or more hook and loop fasteners; or an adhesive tape, or a wire arranged spirally around the plurality of electrical cables.

13. The hospital bed caddy of claim 1, wherein the second compartment is removably coupleable within the housing.

14. A hospital bed caddy, comprising:
a housing comprising:
a base panel having a first opening; and
one or more side panels coupled to the base panel to form a plurality of compartments, the plurality of compartments comprising a first compartment and a second compartment;
a bed rail attachment comprising a hook coupled to or integrally formed with the housing;
a plurality of electrical interfaces arranged in the second compartment;
a cover panel having one or more second openings, the cover panel coupled over the plurality of electrical interfaces to separate the second compartment from the first compartment;
a plurality of electrical cables arranged in the first opening, each electrical cable of the plurality of electrical cables coupled within the second compartment to a corresponding electrical interface of the plurality of electrical interfaces; and
a cable harness or sheath coupled around the plurality of electrical cables.

15. The hospital bed caddy of claim 14, wherein the base panel and the one or more side panels further form a third compartment, of the plurality of compartments, the third compartment arranged adjacent to the first compartment, and wherein a portion of the base panel and at least one front side panel of the one or more side panels of the third compartment further comprise a slot or slot opening.

16. The hospital bed caddy of claim 14, wherein the base panel or at least one side panel of the one or more side panels forming the first compartment or the third compartment has a plurality of perforations.

17. The hospital bed caddy of claim 14, wherein the plurality of electrical interfaces comprise at least one universal serial bus (USB) interface; and wherein the hospital bed caddy further comprises:
a direct current (DC) power supply arranged in the second compartment and coupled to the USB port.

18. The hospital bed caddy of claim 14, wherein the plurality of electrical interfaces comprise of one or more electrical interfaces selected from the group consisting of: a two-pronged alternating current (AC) electrical outlet, a three-pronged AC electrical outlet, a universal serial bus (USB) interface, a registered jack-11 (RJ-11) telecommunications network interface, a Deutsches Institut für Normung (DIN) connector or interface; and combinations thereof.

19. The hospital bed caddy of claim 1, further comprising:
a pivot mount coupled to the housing;
an extension arm coupled to the pivot mount; and
a mobile device mount coupled to the extension arm.

20. The hospital bed caddy of claim 1, further comprising:
at least one lid rotatably coupled to a side panel of the one or more side panels of the plurality of compartments.

21. The hospital bed caddy of claim 1, wherein the cable harness or sheath comprises:
a heat-shrinkable polymeric, tubular sheath; or one or more hook and loop fasteners; or an adhesive tape, or a wire arranged spirally around the plurality of electrical cables.

22. A hospital bed caddy, comprising:
a housing comprising:
a base panel having a first opening; and
one or more side panels coupled to the base panel to form a plurality of compartments, the plurality of compartments comprising a first compartment, a second compartment, and a third compartment, wherein the base panel or at least one side panel of the one or more side panels forming the first compartment or the third compartment has a plurality of perforations, and wherein a portion of the base panel and at least one front side panel of the one or more side panels of the third compartment further comprise a slot or slot opening;
a bed rail attachment comprising a hook coupled to or integrally formed with the housing;
a plurality of electrical interfaces arranged in the second compartment, the plurality of electrical interfaces comprising at least one universal serial bus (USB) interface, wherein the plurality of electrical interfaces are user-accessible from the first compartment;
a direct current (DC) power supply arranged in the second compartment and coupled to the USB port;
a cover panel having one or more second openings, the cover panel coupled over the plurality of electrical interfaces to separate the second compartment from the first compartment;
a pivot mount coupled to the housing;
an extension arm coupled to the pivot mount;
a mobile device mount coupled to the extension arm;
a plurality of electrical cables arranged in the first opening, each electrical cable of the plurality of electrical cables coupled within the second compartment to a corresponding electrical interface of the plurality of electrical interfaces; and
a cable harness or sheath coupled around the plurality of electrical cables.
